(19) United States
(12) Patent Application Publication
(10) Pub. No.: US 2005/0009948 A1
(43) Pub. Date: Jan. 13, 2005

(54) Polyacetal resins with reduced formaldehyde odor

(76) Inventors: Hiroshi Mori, Tochigi (JP); Robert J. Kassal, Newport, DE (US); Kenichi Shinohara, Tochigi (JP)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/867,978

(22) Filed: Jun. 15, 2004

Related U.S. Application Data
(63) Continuation-in-part of application No. 09/852,383, filed on May 10, 2001, now abandoned.
(63) Continuation of application No. 09/287,432, filed on Apr. 7, 1999, now abandoned.

(51) Int. Cl. A61L 2/00

(52) U.S. Cl. 523/102

(57) ABSTRACT

Incorporation of an odor-reducing additive selected from the group consisting of: (i) a low molecular weight primary or secondary amino compound of low volatility, containing at least one amino group and two or more carbon atoms and having a weak basicity of Pkb in the range of 2-8; (ii) succinimide; (iii) anthranilic acid; (iv) 4-amino benzonic acid, and mixtures thereof, into a polyacetal resin to reduce its formaldehyde odor. A combination of the odor-reducing additive with a weak acidic imino was found to have a synergistic effect in further reducing the formaldehyde odor.

### CROSS REFERENCE TO RELATED APPLICATIONS

This application is a continuation-in-part of U.S. application Ser. No. 09/852,383, filed May 10, 2001, which is a continuation of U.S. application Ser. No. 09/287,432, filed on Apr. 7, 1999, which is incorporated as a part hereof.

### BACKGROUND OF THE INVENTION

1. Field of the Invention

The present invention relates to a novel polyacetal resin composition, and articles molded therefrom, which has excellent heat stability, air oven aging and moldability as well as a considerably reduced formaldehyde odor for packaged resins and molded parts.

2. Background

Polyacetal resins, which are prepared by polymerizing a starting material mainly comprising formaldehyde monomer or trioxane, a trimer of formaldehyde, exhibit excellent mechanical and physical properties, such as tensile strength, stiffness, as well as fatigue resistance, sliding resistance, chemical resistance, and the like. The resins are used extensively in various applications as an engineering plastic material due to their excellent physical properties (such as mechanical and electrical properties) and chemical properties. However, the resins at times may evolve traces of formaldehyde from which they were made, even at room temperature. Similarly, molded articles made from polyacetal resins may also evolve traces of formaldehyde, which may make molded parts to be less desirable in some circumstances.

U.S. Pat. No. 5,866,671 discloses polyacetal compositions containing acidic hydantoin and imidazole derivatives with reduced odor levels.

There is still a demand for means to reduce the formaldehyde odor in polyacetal resins as well as that which may arise in articles molded from polyacetal resins.

### SUMMARY OF THE INVENTION

The present inventors have surprisingly found a novel composition comprising:
a) a polyacetal resin; and
(b) one or more members of the group of odor-reducing additives consisting of
   (i) succinimide; and
   (ii) a low molecular weight primary or secondary amino compound of low volatility, containing at least one amino group and two or more carbon atoms, and having a pKb in the range of about 2-8, excepting and excluding those amino compounds described by the formula 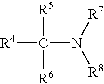
wherein R⁴, R⁵ and R⁶ are selected from the class consisting of hydroxyalkyl groups having 1˜4 carbon atoms and alkyl groups having 1˜9 carbon atoms, and the R⁷ and R⁸ groups are selected from the class consisting of hydrogen and alkyl groups having 1˜4 carbon atoms; wherein the composition is characterized by a formaldehyde concentration at room temperature that is less than about 50% of the formaldehyde concentration of the polyacetal resin itself.

The invention also relates to a novel composition comprising: a) a polyacetal resin; a sufficient amount of at least b) an odor-reducing additive selected from the group consisting of: (i.) a low molecular weight primary or secondary amino compound of low volatility, containing at least one amino group and two or more carbon atoms and having a weak basicity of Pkb in the range of 2-8; (ii) succinimide; (iii) anthranilic acid; (iv) 4-amino benzonic acid; and c) a weak acidic organic cyclic compound having an active imino, to provide a synergistic effect in reducing the formaldehyde odor of at least in half.

Pursuant to another aspect of the present invention, there is a polyacetal resin composition consisting essentially of (a) a polyacetal resin, and (b) a low molecular weight primary amino compound of low volatility, containing at least one amino group and two or more carbon atoms, and having a pKb in the range of about 2-8; wherein the composition is characterized by a formaldehyde concentration at room temperature that is less than about 50% of the formaldehyde concentration of the polyacetal resin itself and wherein the amino compound is ethyl p-aminobenzoate.

Pursuant to another aspect of the present invention, there is a method for reducing the formaldehyde concentration of a part molded from a polyacetal resin, comprising
(a) forming a composition consisting essentially of (i) the polyacetal resin, and (ii) one or more members of the group of odor-reducing additives consisting of
(A) succinimide; and
(B) a low molecular weight primary or secondary amino compound of low volatility, containing at least one amino group and two or more carbon atoms, and having a pKb in the range of 2-8; excepting and excluding those amino compounds described by the formula 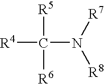
   wherein R⁴, R⁵ and R⁶ are selected from the class consisting of hydroxyalkyl groups having 1˜4 carbon atoms and alkyl groups having 1˜9 carbon atoms, and the R⁷ and R⁸ groups are selected from the class consisting of hydrogen and alkyl groups having 1˜4 carbon atoms; wherein the amino compound of low volatility is characterized by T_{bp}>Tₘ−60° C., where T_{bp} is the boiling point of the amino compound and Tₘ is the melting point of the polyacetal resin; and wherein the composition is characterized by a formaldehyde concentration at room temperature that is less than about 50% of the formaldehyde concentration of the polyacetal resin itself; and
(b) molding the part from the composition.

Pursuant to another aspect of the present invention, there is a method for reducing the formaldehyde concentration of a part molded from a polyacetal resin, comprising
(a) forming a composition consisting essentially of (i) the polyacetal resin, and (ii) a low molecular weight primary amino compound of low volatility, containing at least one amino group and two or more carbon atoms, and having a pKb in the range of 2-8; wherein the composition is characterized by a formaldehyde concentration at room temperature that is less than about 50% of the formaldehyde concentration of the polyacetal resin itself and wherein the amino compound is ethyl p-aminobenzoate; and
(b) molding the part from the composition.

### DETAILED DESCRIPTION OF THE INVENTION

Polyacetal Resins

The polyacetal base resin that may be used in the compositions of the present invention is a high-molecular weight polymer comprised of repeating oxymethylene units (—CH2O—) which may be selected from among polyoxymethylene homopolymers, copolymers (including block copolymers) and terpolymers comprising oxymethylene units and a minor amount of other constituent units.

The polyacetals used in the compositions of the present invention may, moreover, be linear, branched or crosslinked, with terminal groups thereof being either not protected or being protected. The polyacetal resin will generally have a number average molecular weight in the range of 5,000 to 100,000, preferably 10,000 to 70,000. The molecular weight can conveniently be measured by gel permeation chromatography in m-cresol at 160° C. using a Du Pont PSM bimodal column kit with nominal pore size of 60 and 1000 Angstrom.

The polyacetal used in the present invention can be either a homopolymer, a copolymer or a mixture thereof. The preferred homopolymer is generally prepared by the polymerization of anhydrous formaldehyde or a cyclic trimer thereof, i.e., trioxane. Generally, the homopolymer is stabilized against thermal decomposition by end-capping with a suitable moiety having greater stability as compared to the oxymethylene units. The preferred copolymer on the other hand is a high-molecular weight polymer comprising between about 85 to 99.9% of repeating oxymethylene units randomly interspersed with higher oxyalkylene units (e.g., having two or more adjacent carbon atoms).

Copolymers can contain one or more comonomers, such as those generally used in preparing polyacetal compositions. Comonomers more commonly used include alkylene oxides of 2-12 carbon atoms and their cyclic addition products with formaldehyde. The quantity of comonomer will not be more than 20 weight percent, preferably not more than 15 weight percent, and most preferably about 2 weight percent. The most preferred comonomer is ethylene oxide. Generally polyacetal homopolymer is preferred over copolymer because of its greater stiffness. Preferred polyacetal homopolymers include those whose terminal hydroxyl groups have been end-capped by a chemical reaction to form ester or ether groups, preferably acetate or methoxy groups, respectively.

Additives

The odor-reducing additives of the present invention include: i) a water-soluble, low molecular weight primary or secondary amino compound, containing at least one amino group and two or more carbon atoms and having a weak basicity of Pkb in the range of 2-8; (ii) succinimide; (iii) anthranilic acid; (iv) 4-amino benzonic acid, and mixtures thereof.

With respect to the water-soluble, low molecular weight primary or secondary amino compounds containing at least one amino group and two or more carbon atoms, no particular limitation is imposed on the number of amino group(s). The amino compounds can be an aliphatic, alicyclic, aromatic or heterocyclic group having two or more carbon atoms. The amino compounds may contain one or more groups other than amino group(s), for example, hydroxyl, ester, ether, carboxyl, carbonyl, amido, imido, sulfonic, carboxamido, imino and/or unsaturated groups.

The water-soluble, low molecular weight primary or secondary amino compounds are characterized by being weak bases, i.e., having have a basicity pkb's ranging from about 2 to 8, preferably being very weak bases, i.e., having have a basicity pkb's ranging from about 4 to 8.

The water-soluble, low molecular weight primary or secondary amino compounds are further characterized as having a low volatility, i.e., the boiling point of the amino compounds should be as follows: T_{bp}>Tₘ−60 C, where T_{bp} is the boiling point of the amino compounds and Tₘ is the melting point of the polyacetal base resin.

Specific examples of such water-soluble, low molecular weight primary or secondary amino compounds include, but are not limited to, e.g., monoethanolamine, diethanolamine, tris(hydroxymethyl)aminomethane, alkyl aminobenzoates such as ethyl p-aminobenzoate, methyl anthranylate and butyl m-aminobenzoate, 2-amino-2-ethyl-propanediol and 2-amino-2-methyl-propanol.

Tris(hydroxymethyl)aminomethane, ethyl p-aminobenzoate, 2-amino-2-ethyl-propanediol and 2-amino-2-methyl-propanol are preferred for use as water-soluble, low molecular weight primary or secondary amino compounds in accordance with this invention. Tris(hydroxymethyl)aminomethane and ethyl p-aminobenzoate are most preferred.

The amount of the odor-reducing additives to added to the compositions of the present invention should be in a sufficient amount to reduce the formaldehyde level of the resin or its molded parts in half. The amount of additives is about 0.01-10 in parts by weight, preferably 0.02-5 by weight, and most preferably 0.05-2 parts by weight, per 100 parts by weight of the polyacetal resin.

Other Additives

The composition of the present invention can further contain, one or more ordinary additives including: lubricants; nucleating agents; mold release agents; antistatic agents; surfactants; organic polymeric materials; inorganic, organic, fibrous, granular or platy fillers, anti-oxidants, pigments, colorants, carbon black, reinforcing agents and fillers such as a glass fiber, etc., in such an amount as not to negate the effects of this invention.

Representative lubricants that may be used include, but are not limited to, silicone types such as dimethylpolysiloxanes and their modifications; oleic acid amides; alkyl acid amides, e.g., stearic acid amide types; bis-fatty acid amid type lubricants such as bisamides; non-ionic surfactant type lubricants; hydrocarbon type lubricants waxes, chlorohydrocarbons, fluorocarbons; fatty acid type lubricants including oxy-fatty acid; ester-type lubricants including lower alcohol esters of fatty acids; alcohol type lubricants including polyvalents, polyglycols, polyglycerols; and metal soaps such as lauric acid, stearic acid, etc., with metals.

Also, it is desirable to add antioxidant to prevent oxidative deterioration of the resin. Hindered phenol type antioxidants are preferred, and those antioxidants with melting point higher than 100° C., especially above 120° C., are most preferred.

It is also desirable to add heat stabilizers to the composition of this invention as formaldehyde scavengers such as: polyamide resins, amide compounds, urea derivatives and triazine derivatives.

Suitable polyamide resins include polyamide compounds, especially nylon terpolymers, hydroxy containing polymers, and nonmelting nitrogen or hydroxy containing compounds, e.g., polyamide 6, polyamide 6/12 copolymer, polyamide 6/66/610 terpolymer, polyamide 6/66/612 terpolymer, ethylene-vinyl alcohol copolymer, acrylamide (co)polymer, acrylamide/N,N-methylenebis-acrylamide copolymer, stearic acid monoglyceride and poly beta alanine and the like.

Suitable amide compounds are stearic acid amide, oleic acid amide, erucic acid amide, ethylenediamine-distearic acid amide, ethylenediamine-dibehenic acid amide, hexamethylenediamine-distearic acid amide, ethylenediamine-dioleic acid amide, ethylenediamine-dierucic acid amide, xylylenediamine-dierucic acid amide, di(xylylenediamine-stearic acid amide)sebacic acid and the like.

Suitable urea derivatives are N-phenylurea, N,N′-diphenylurea, N-phenylthiourea, N,N′-diphenylthiourea and the like.

Suitable triazine derivatives are melamine, benzoguanamine, N-phenylmelamine, N,N′-diphenylmelemine, N-methylolmelamine, N,N′-trimethylolmelamine, 2,4-diamino-6-cyclohexyltriazine, and the like.

These heat stabilizer may be used individually or in combination. Of these, nylon 66, poly beta-alanine, ethylenediamine-distearic acid amide, ethylenediamine-dibehenic acid amide, ethylenediamine-dierucic acid amide, di(xylylenediamine-stearic acid amide)sebacic acid amide are preferred.

The composition may also contain an organic cyclic compound having an active imino group according to the formula: 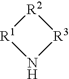
wherein R¹, R² and R³ represent divalent organic radicals. The organic cyclic compound having an active imino group may be used in an amount sufficient for the composition to have a formaldehyde concentration at room temperature of about less than 50% of the formaldehyde concentration of a polyacetal composition free of the odor-reducing additive described above and the organic cyclic compound having an active imino group.
Preparation

The compositions of the present invention can be prepared by any means of compounding. The additives can be added as dry powders, as concentrates (“master-batch”), as dispersions, or as solutions. One preferred method of incorporation involves adding the odor-reducing additives to polyacetal resin pellets to coat the pellets, and thereby mixing the mixtures in an extruder or injection molder. In addition, the odor-reducing additives may be compounded into the polyacetal resin at the same time that other additives, such as thermal stabilizers, antioxidants, fillers, etc., are compounded therein. The additives may be added in the form of particle or in the molten state.

Molded parts of these polyacetal resin compositions may be formed by any molding process conventional in the plastics-forming art, including compression molding, vacuum forming, injection molding, extrusion molding, blow molding, rotary molding, melt spinning, and heat molding. Injection molding is especially preferred. During injection molding of the claimed compositions, mold deposits attached to the mold were evaluated visually and were hardly observed or not found at all.

### EXAMPLES

In the Examples and Comparative Examples, the characteristics of the acetal resin compositions and moldings were determined as follows:

Thermal Stability Test. Two grams of resin pellets were melted for 30 minutes at 250° C. in a nitrogen atmosphere. The formaldehyde gas generated by the decomposition of the resin was introduced into a 4% aqueous sodium bisulfite solution, followed by titration with a 0.1 N hydrochloric acid (HCl). The amount of formaldehyde gas generated (TEF) is expressed by the following formula:
Amount of formaldehyde formed(%)=30.03NV/S×100
wherein V was the amount of HCl, in ml, required for titrating after 30 minutes, N was the normality, in ml, of HCl, S was the amount of sample, in grams, and 30.03 was the molecular weight of formaldehyde.

Odor Test 1—Pellets: 100 g of resin pellets were sealed in a 300 ml polyethylene containers and kept at various temperatures: room temperature, 50° C. and 80° C. At the start of the test, then after 1 and 2 hours respectively, 10 ml of gas was removed from the containers using a syringe and formaldehyde concentration was measured using a gas-phase measuring device made by Lion Company called Formaldemeter Mark II.

Odor Test 2—Pellets: Resin pellets were stored at room temperature in sealed 50-lb. bags made out of polyethylene. Gas samples were tested by removing 1 cc. gas from the sealed bags and formaldehyde concentration was measured using the gas-phase measuring device Formaldemeter Mark II.

Odor Test 3—Molded Tensile-bar tests: 1/16″ Tensile-bars were molded from a control composition as well as the claimed compositions. The tensile bars were stored at room temperature in sealed 1 gallon polyethylene containers. Gas samples were tested by removing 1 cc. gas from the sealed containers and formaldehyde levels were measured using a Formaldemeter Mark II.

Odor Test 4—Molded part test: molded parts were molded from the claimed compositions, kept in sealed 300 ml polyethylene containers at various temperatures: 50° C. and 80° C. Gas samples were tested by removing 1 cc. gas from the sealed containers and formaldehyde levels were measured using a Formaldemeter Mark II by the Lion Company.

Components: The polyacetal resins used in the Examples below were:
1. Polyacetal A available from E. I. du Pont de Nemours and Company of Wilmington, Del. USA (“DuPont”), having a number average molecular weight of about 37,000;
2. Polyacetal B available from DuPont, having a number average molecular weight of about 30,000;
3. Polyacetal C also available from DuPont, having a number average molecular weight of about 77,000; and
4. Polyacetal D, available from Polyplastics, Japan, under the tradename Duracon® M25.

The additives used in the Examples are as listed in the tables below. In the Examples, THAM is tris(hydroxymethyl)aminomethane, EPA is ethyl p-aminobenzoate, AEPD is 2-amino-2-ethyl-propanediol and AMP is 2-amino-2-methyl-propanol.

Preparations: The polyacetal resin was mixed with the additives in an extruder and the resultant resin was pelletized, and in some instances, formed into molded parts. The samples were evaluated by the Thermal Heat Stability test and the various Odor Tests. Comparative Examples were those obtained by processing similar polyacetal without any odor-reducing additives at all.

The results of evaluation are listed in the tables below, demonstrating that the examples using the additives of the present invention were able to suppress the formaldehyde odors in resin without severely adversely affecting the melt stability of the resins.

In the first set of experiments, polyacetal B was used and the formaldehyde levels were measured using the Thermal Stability test as well as the Odor Test 1 (pellets in 300 ml PE bottle):

TABLE 1

| | | | | ppm | ppm | ppm |
|---|---|---|---|---|---|---|
| | | | | CHCHO | CHCHO | CHCHO |
| Exam- | Wt. % | | | ppm | 50° C./ | 80° C./ |
| ple | additive | Additive | TEF | initial | 1 hr | 1 hr |
| Com | — | None | 0.15 | 19.5 | 84 | >85 |
| 1-1 | | | | | | |
| 1-2 | 0.05 | EPA | 0.13 | 6.8 | 30.9 | 75.4 |
| 1-3 | 0.2 | EPA | 0.12 | 0.5 | 3.3 | 13.8 |
| 1-4 | 0.5 | EPA | 0.13 | 0.3 | 1.3 | 7.3 |
| 1-5 | 0.2 | THAM | 0.23 | 0.5 | 1.6 | 3.0 |

In the next set of experiments, polyacetal A was used and the formaldehyde levels were measured using the Thermal Stability test as well as the Odor Test 2 (50 lb. PE sealed bags).

TABLE 2

| | | | | Ppm | Ppm | ppm |
|---|---|---|---|---|---|---|
| | | | | CHCHO | CHCHO | CHCHO |
| | Wt. % | | | 19° C./3 | 23° C./7 | 24° C./23 |
| Example | additive | Additives | TEF | day | day | day |
| Compare | 0.0 | None | 0.47 | 34.4 | 56.0 | 44.8 |
| 2-1 | | | | | | |
| 2-2 | 0.2 | THAM | 0.83 | 0.3 | 0.4 | 0.4 |
| 2-3 | 0.2 | Theophyline | 2.59 | 2.9 | 0.7 | 1.5 |
| 2-4 | 0.2 | Succinimide | 1.12 | 0.9 | 1.1 | 1.2 |
| 2-5 | 0.2 | Anthranilamide | 1.33 | 0.3 | 0.3 | 0.5 |
| 2-6 | 0.2 | Glycine | — | 7.6 | 12.0 | 15.1 |
| | | anhydride | | | | |
| 2-7 | 0.2 | Anthranilic acid | 2.37 | 0.3 | 1.2 | 2.0 |
| 2-8 | 0.2 | 4-amino | — | 1.6 | 5 | 13.1 |
| | | benzoic acid | | | | |
| 2-9 | 0.2 | Uracil | 0.92 | 1.7 | 3.2 | 6.1 |
| 2-10 | 0.2 | Barbituric acid | — | 0.3 | 0.5 | 0.5 |

In the third set of experiments, polyacetals C and D were used and the formaldehyde levels were measured via Odor Tests 1 (pellets in 300 ml PE bottle):

TABLE 3

| | | | | ppm | ppm | |
|---|---|---|---|---|---|---|
| | | | | CH2O | CH2O | ppm |
| Exam- | Polyacetal | Wt. % | Addi- | 23 C./ | 50 C./ | CH2O |
| ple | Resin | Additive | tive | 1 hr | 1 hr | 80 C./1 hr |
| Com | C | — | None | 12.7 | 70.8 | >86 |
| 3-1 | | | | | | |
| 3-2 | C | 0.05 | THAM | 0.4 | 1.7 | 1.0 |
| 3-3 | C | 0.05 | AMP | 1.2 | 12.2 | 63.8 |
| 3-4 | C | 0.05 | AEPD | 0.4 | 2.0 | 2.8 |
| 3-5 | D | 0.05 | TRAM | 0.1 | 1.9 | 2.0 |

In table 4, polyacetal A was used and the formaldehyde levels were measured via Odor Tests 2 (50 lb. sealed bags) and 3 (tensile-bar samples). In some of the examples, an acidic organic cyclic compound having an active imino such as 5,5-dimethylhydantoin (DMH) was added to the additive of the present invention.

The combination of the odor-reducing additives of the invention with the weak acidic imino was found to have a synergistic effect in reducing formaldehyde odor:

TABLE 4

| | | | | | | | ppm |
|---|---|---|---|---|---|---|---|
| | | | | ppm | ppm | ppm | CH2O |
| Exam- | Wt. % | Addi- | | CH2O | CH2O | CH2O | T-Bars |
| ple | additive | tive | TEF | 3 days | 7 days | 21 days | 1 day |
| Com | — | None | 0.69 | 34.4 | 95.6 | 80 | 383.2 |
| 4-1 | | | | | | | |
| 4-2 | 0.05 | DMH | 0.63 | 5.1 | 5.3 | 3.8 | 147 |
| 4-3 | 0.1 | DMH | 0.56 | 5.2 | 3.3 | 3 | 25.6 |
| 4-4 | 0.2 | DMH | 0.64 | 2.6 | 1.8 | 1.8 | 16 |
| 4-5 | 0.2 | THAM | — | — | 0.4 | 0.4 | 16 |
| 4-6 | 0.1 | 50% | 0.85 | 0.3 | 0.3 | 0.4 | 12.6 |
| | | THAM/ | | | | | |
| | | 50% | | | | | |
| | | DMH | | | | | |
| 4-7 | 0.2 | 50% | 0.73 | 0.3 | 0.3 | 0.3 | 7.1 |
| | | THAM/ | | | | | |
| | | 50% | | | | | |
| | | DMH | | | | | |

In another set of examples, polyacetal B was used and the formaldehyde levels were measured using Odor Test 1 (pellets in PE bottles):

TABLE 5

| | | | ppm | | ppm |
|---|---|---|---|---|---|
| | | | CH2O | ppm | CH2O |
| | Wt. % | | 23 C./ | CH2O | 80 C./ |
| Example | additive | Additive | 1 hr | 50 C./1 hr | 1 hr |
| Compare | — | None | 19.5 | 84 | >85 |
| 5-1 | | | | | |
| 5-2 | 0.05 | ethyl | 6.8 | 30.9 | 75.4 |
| | | p-aminobenzoate | | | |
| 5-3 | 0.2 | ethyl | 0.5 | 3.3 | 13.8 |
| | | p-aminobenzoate | | | |
| 5-4 | 0.5 | ethyl | 0.3 | 1.3 | 7.3 |
| | | p-aminobenzoate | | | |
| 5-5 | 0.2 | DMH | 0.8 | 3.9 | 16.6 |
| 5-6 | 0.2 | THAM | 0.5 | 1.6 | 3.0 |
| 5-7 | 0.1 | THAM | 0.5 | 1.4 | 3.3 |
| 5-8 | 0.1 | 50% THAM/ | 0.5 | 4.7 | 11.7 |
| | | 50% DMH | | | |

The examples were repeated with 30 g of molding parts from polyacetal B, and the formaldehyde levels were measured using Odor Test 4 (molded parts in 300 ml PE bottle), confirming the synergistic effect of a combination of the odor-reducing additives of the invention with a weak acidic organic cyclic compound having an active imino such as 5,5-dimethylhydantoin (DMH):

TABLE 6

| | | | | ppm |
|---|---|---|---|---|
| | | | | CH2O |
| | Wt. | | Ppm CH2O | 80 C./ |
| Example | % additive | Additive | 50 C./1 hr | 1 hr |
| Compare | — | None | 53.8 | >85 |
| 6-1 | | | | |
| 6-2 | 0.05 | Ethyl p-aminobenzoate | 58.7 | >85 |
| 6-3 | 0.2 | Ethyl p-aminobenzoate | 45.2 | 60.9 |
| 6-4 | 0.5 | Ethyl p-aminobenzoate | 35.7 | 32.4 |
| 6-5 | 0.2 | DMH | 30.6 | 42.9 |
| 6-6 | 0.2 | THAM | 67.1 | >85 |
| 6-7 | 0.1 | THAM | 83 | >85 |
| 6-8 | 0.1 | 50% THAM/50% 5,5- | 10.4 | 38.1 |
| | | DMH | | |

As is apparent from the foregoing description, the materials prepared and procedures followed relate only to specific embodiments of the broad invention. While forms of the invention have been illustrated and described, modifications can be made without departing from the spirit and scope of the invention. Accordingly, it is not intended that the invention be limited thereby.

1. A polyacetal resin composition consisting essentially of (a) a polyacetal resin, and (b) one or more members of the group of odor-reducing additives consisting of
(i) succinimide; and
(ii) a low molecular weight primary or secondary amino compound of low volatility, containing at least one amino group and two or more carbon atoms, and having a pKb in the range of about 2-8, excepting and excluding those amino compounds described by the formula 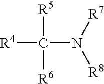
wherein R⁴, R⁵ and R⁶ are selected from the class consisting of hydroxyalkyl groups having 1˜4 carbon atoms and alkyl groups having 1˜9 carbon atoms, and the R⁷ and R⁸ groups are selected from the class consisting of hydrogen and alkyl groups having 1˜4 carbon atoms;
wherein the composition is characterized by a formaldehyde concentration at room temperature that is less than about 50% of the formaldehyde concentration of the polyacetal resin itself.

2. A composition according to claim 1 wherein the amino compound has a pKb in the range of about 4-8.

3. A composition according to claim 1 wherein the amino compound is characterized by T_{bp}>Tₘ−60° C., where T_{bp} is the boiling point of the amino compound and Tₘ is the melting point of the polyacetal resin.

4. A composition according to claim 1 wherein the amino compound is selected from the group consisting of diethanolamine, ethyl p-aminobenzoate, methyl anthranylate, butyl m-aminobenzoate, and mixtures thereof.

5. A polyacetal resin composition consisting essentially of (a) a polyacetal resin, and (b) a low molecular weight primary amino compound of low volatility, containing at least one amino group and two or more carbon atoms, and having a pKb in the range of about 2-8; wherein the composition is characterized by a formaldehyde concentration at room temperature that is less than about 50% of the formaldehyde concentration of the polyacetal resin itself and wherein the amino compound is ethyl p-aminobenzoate.

6. A composition according to claim 1 wherein the amino compound is present in the composition in an amount of about 0.01˜10 parts by weight, per 100 parts by weight of the polyacetal resin.

7. A composition according to claim 1 wherein the polyacetal resin is an acetal copolymer.

8. A composition according to claim 1 further consisting essentially of an organic cyclic compound having an active imino group according to the formula 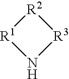
wherein R¹, R² and R³ are divalent organic radicals.

9. A composition according to claim 1 further consisting essentially of at least one additive selected from the group consisting of nucleating agents, mold release agents, surfactants, impact modifiers, reinforcing agents, anti-static agents, plasticizers, lubricants, fillers and colorants.

10. A shaped article produced from a composition according to claim 1.

11. A method for reducing the formaldehyde concentration of a part molded from a polyacetal resin, comprising
(a) forming a composition consisting essentially of (i) the polyacetal resin, and (ii) one or more members of the group of odor-reducing additives consisting of
(A) succinimide; and
(B) a low molecular weight primary or secondary amino compound of low volatility, containing at least one amino group and two or more carbon atoms, and having a pKb in the range of 2-8; excepting and excluding those amino compounds described by the formula 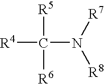
wherein R⁴, R⁵ and R⁶ are selected from the class consisting of hydroxyalkyl groups having 1˜4 carbon atoms and alkyl groups having 1˜9 carbon atoms, and the R⁷ and R⁸ groups are selected from the class consisting of hydrogen and alkyl groups having 1˜4 carbon atoms; wherein the amino compound of low volatility is characterized by T_{bp}>Tₘ−60° C., where T_{bp} is the boiling point of the amino compound and Tₘ is the melting point of the polyacetal resin; and
wherein the composition is characterized by a formaldehyde concentration at room temperature that is less than about 50% of the formaldehyde concentration of the polyacetal resin itself; and
(c) molding the part from the composition.

12. A method according to claim 11 further comprising the step of selecting as the amino compound a member of the group consisting of diethanolamine, ethyl p-aminobenzoate, methyl anthranylate, butyl m-aminobenzoate, and mixtures thereof.

13. A method for reducing the formaldehyde concentration of a part molded from a polyacetal resin, comprising
(a) forming a composition consisting essentially of (i) the polyacetal resin, and (ii) a low molecular weight primary amino compound of low volatility, containing at least one amino group and two or more carbon atoms, and having a pKb in the range of 2-8; wherein the composition is characterized by a formaldehyde concentration at room temperature that is less than about 50% of the formaldehyde concentration of the polyacetal resin itself and wherein the amino compound is ethyl p-aminobenzoate; and
(b) molding the part from the composition.
